(19) United States
(12) Patent Application Publication
(10) Pub. No.: US 2010/0239596 A1
(43) Pub. Date: Sep. 23, 2010

(54) GRP78 AND TUMOR ANGIOGENESIS

(75) Inventors: Amy S. Lee, San Marino, CA (US); Florence Hofman, Venice, CA (US)

Correspondence Address: FISH & RICHARDSON P.C., P.O BOX 1022, Minneapolis, MN, 55440-1022, US

(73) Assignee: UNIVERSITY OF SOUTHERN CALIFORNIA, Los Angeles, CA (US)

(21) Appl. No.: 12/674,583

(22) PCT Filed: Aug. 22, 2008
(86) PCT No.: PCT/US08/73980

§ 371 Date: Mar. 5, 2010

Related U.S. Application Data
(60) Provisional application No. 60/957,269, filed on Aug. 22, 2007.

(51) Int. Cl. A61K 39/395; A61K 31/713; A61K 31/352; A61K 31/60; A61K 31/704; A61K 38/14; A61P 35/00

(52) U.S. Cl. 424/172.1; 514/44 A; 514/456; 514/159; 514/34; 514/3.1

(57) ABSTRACT

Methods of preventing or reducing tumor angiogenesis in a subject, comprising administering to the subject one or more agents that inhibit expression or activity of GRP78 are provided. Also provided are methods of sensitizing tumor blood vessels to a chemotherapeutic agent comprising administering to the subject one or more agents that inhibit expression or activity of GRP78. Provided is also a method of reducing tumor microvessel density in a subject, comprising selecting a subject with a tumor, wherein the subject is in need of reduction of tumor microvessel density, and administering to the subject one or more agents that inhibit expression or activity of GRP78.

### CROSS REFERENCE TO RELATED APPLICATIONS

This application claims priority to U.S. Provisional Application No. 60/957,269, filed Aug. 22, 2007, which is incorporated by reference herein in its entirety.

### STATEMENT REGARDING FEDERALLY FUNDED RESEARCH

The present invention was made with support from Grant Nos. CA027607 and CA111700 from the National Institutes of Health. The U.S. government has certain rights in this invention.

### BACKGROUND

BRP78/BiP, an endoplasmic reticulum (ER) chaperone protein, is required for proper folding and assembly of membrane and secretory proteins. GRP78 is upregulated under stress conditions such as ER stress, glucose deprivation, hypoxia or the presence of toxic agents. The ER is an essential cellular organelle where secretory and membrane proteins are synthesized and modified, and is also a major intracellular Ca2+ storage compartment. The ER is also thought to be a regulator of apoptosis. The unfolded protein response (UPR) triggers multiple pathways to allow cells to respond to endoplasmic reticulum (ER) stress. The UPR can be protective through activation of adaptive, anti-apoptotic pathways as well as commit cells to undergo apoptosis under sever stress. Cancer cells exhibit elevated glucose metabolism and are often exposed to tumor hypoxia, resulting in ER stress. However, the precise role of UPR in the development of tumors remains unclear. Tumor growth and survival is dependent on the supply of nutrients and oxygen provided by blood vessels within the cancer; thus the tumor vasculature is essential for tumor growth and survival and is a key target for anticancer therapy.

### SUMMARY

Methods of preventing or reducing tumor angiogenesis in a subject are provided. The method includes administering to the subject one or more agents that inhibit expression or activity of GRP78 are provided. Also provided are methods of sensitizing tumor blood vessels to a chemotherapeutic agent comprising administering to a subject one or more agents that inhibit expression or activity of GRP78. For example, provided herein is a method that includes selecting a subject with a tumor, wherein the cells of the blood vessels of the tumor are resistant to one or more chemotherapeutic agents and administering to the subject one or more agents that inhibits the expression or activity of GRP78.

Provided is also a method of reducing tumor microvessel density in a subject. The method includes selecting a subject with a tumor, wherein the subject is in need or reduction of tumor microvessel density, and administering to the subject an agent that inhibit expression or activity of GRP78.

### DESCRIPTION OF DRAWINGS

FIGS. 1A-C show the overexpression of GRP78 protein in tumor-associated brain endothelial cells (TuBEC) and tumor vasculature. FIG. 1A shows images of an immunostain for GRP78 of cyto-centrifuge cell preparations of primary cultures of TuBEC and normal human brain endothelial cells (BEC). FIG. 1B (top panel) is a Western blot for GRP78 of two representative patient samples of TuBEC and BEC. The bottom panel is a graph showing relative GRP78 protein expression levels as compared to β-actin. FIG. 1C shows images of immunostains of glioma and normal brain tissues. In the upper panels, frozen sections of glioma tissue were stained with anti-GRP78 antibody, anti-CD31 antibody, and DAPI nuclear staining; the images were merged in the last panel. The arrows designate blood vessels. In the lower panels, cryostat sections of normal brain tissues were immunostained similarly with anti-GRP78 antibody, anti-CD31 antibody, and DAPI nuclear staining; the images were merged in the last panel. Bar=100 microns.

FIGS. 2A-C show that chemoresistance is reversed in TuBEC with reduced GRP78 protein. FIG. 2A is a graph showing the percent cell viability using the MTT assay for TuBEC and BEC that were exposed to 1, 10, 50 μM etoposide (Eto), and vehicle control (DMSO) for 72 hours. Vehicle treatment served as 100% viable control. FIG. 2B shows images of an immunostain for GRP78 14 days post-transfection, on TuBEC infected with control siRNA (siCtr1A) or siRNA specifically targeted against human GRP78 (siGRP78A). 200× magnification. FIG. 2C is a graph showing cell death using the cell death ELISA assay for TuBEC infected with lentivirus siCtr1A, lentivirus siGRP78A constructs, or uninfected and treated five days post-infection with control media, CPT-11 [100 μM], Eto [50 μM] or temozolomide (TMZ) [300 μM] for another 7 days. The * represents p<0.05.

FIGS. 3A-D show TuBEC with reduced GRP78 are susceptible to caspase-dependent apoptotic cell death when treated with chemotherapeutic agents. FIG. 3A shows images of an immunostain for GRP78 in TuBEC treated with a second lentivirus containing siRNA targeted against human GRP78 (siGRP78B). TuBEC infected with siGRP78B show a reduction in GRP78 expression. TuBEC infected with control siRNA (siCtr1B) did not affect GRP78 expression. 400× magnification. FIG. 3B is a graph showing percent cell death using the cell death ELISA assay for uninfected TuBEC or cultures infected with siCtr1B or siGRP78B that were treated with media or drugs (CPT-11 [100 μM], Eto [50 μM] or TMZ [300 μM]) alone, or incubated with the caspase inhibitor (Q-VDOPH) [10 μM] for 7 days. FIG. 3C is a graph showing percent apoptotic cells using the TUNEL assay for TuBEC uninfected or infected with lentivirus expressing siCtr1A or lentivirus expressing siGRP78B for five days, followed by treatment with Eto [150 μM] for 7 days. Apoptotic death was calculated as percent positive in drug treated compared to cells incubated in media alone. FIG. 3D is a graph showing percent cell death for TuBEC treated with EGCG (20 μM) alone or in combination with TMZ (300 μM), CPT-11 (100 μM), or Eto (50 μM) for 7 days. Media containing DMSO served as the vehicle control. The * represents p<0.05.

FIGS. 4A-C shows overexpression of GRP78 in BEC promotes chemoresistance. FIG. 4A is a Western blot for GRP78 and GAPDH, five days post-infection, on lysates from BEC that are uninfected (UI) or infected with lentivirus expressing either green fluorescence protein (GFP) or GRP78. FIG. 4B is a graph showing percent cell death using the cell death ELISA assay for BEC that were uninfected or infected with lentivirus GFP or GRP78 and treated with Eto [50 μM] for 5 or 7 days. Percent death was calculated based on media treatment of uninfected cells. FIG. 4C is a graph showing percent cell death using the cell death ELISA assay for BEC that were uninfected or infected with lentivirus GFP or GRP78 and treated with CPT-11 [50 μM] for 5 or 7 days. Percent death as calculated based on the total cell death control. The * represents p<0.05.

FIGS. 5A-D show the characterization of the Grp78 heterozygous mice. FIG. 5A is a graph showing the mean fasting body weight of male and female cohorts (n=7 to 12) of WT (+/+) and Grp78 heterozygous (+/−) siblings plotted against their age in weeks. The bars represent standard error of the mean. The insert shows a Western blot of GRP78 and β-actin levels in total cell lysates from the liver of 30 week old Grp78 WT and heterozygous siblings. FIG. 5B are images of a H&E staining of paraffin sections of the organs from 15 week old female Grp78 WT and heterozygous mice: brain, Br, heart, Ht; liver, Li; Spleen, Sp; kidney, Ki and pancreas, Pa. The bars=100 μm. FIG. 5C is a graph showing levels of pre-immune serum IgM and IgG subtypes in Grp78+/+ (n=8) and +/−(n=8) siblings. The distribution was shown with the bar indicating the average level. FIG. 5D is a graph showing relative levels of TNP-specific IgG subtypes after immunization with the antigen TNP-KLH in Grp78+/+ (n=7) and +/−(n=8) siblings.

FIGS. 6A-C show that Grp78 heterozygosity prolongs the latency period and suppresses tumor growth. FIG. 6A is a summary of the time of appearance of the primary tumor of the indicated 4 tumor genotypes. FIG. 6B is a graph showing tumor size in cohorts of female Grp78+/+, PyT mice (n=15) and Grp78+/+, PyT mice (n=15) monitored for the time of appearance and size of the primary tumor in each mouse. The dots represent the observed mean tumor volumes and the lines display the model-predicted tumor volumes. The bars represent 95% CI for the mean tumor volume at week 15. The likelihood ratio test based on random coefficient model comparing the two curves showed that significant reduction of tumor growth in heterozygous mice compared to the WT mice (p<0.001). FIG. 6C shows images comparing the organ size and morphology harvested from mice of the 4 indicated genotypes: brain, Br, lung, Lu; heart, Ht; liver, Li; spleen, Sp; kidney, Ki; and pancreas, Pa.

FIGS. 7A-D show tumors from Grp78 heterozygous mice exhibit reduced proliferation and increased apoptosis. FIG. 7A shows images of a hematoxylin and eosin (H&E) staining of paraffin-embedded tumor sections of WT and heterozygous mice. Two representative staining patterns are shown for each tumor genotype. FIG. 7B shows images of an immunofluorescent double stain for anti-keratin and anti-vimentin of Paraffin-embedded tumor sections from the Grp78 WT and heterozygous mice. The nuclei were counterstained with DAPI. Two representative merged staining patterns are shown for each tumor genotype. FIG. 7C shows images of an immunohistochemical (MC) staining of paraffin-embedded tumor sections from the Grp78 WT and heterozygous mice with antibodies against GRP78 (upper panel), and PCNA (lower panel), and lightly counterstained with hematoxylin. FIG. 7D shows images of a TUNEL assay for cell death on paraffin-embedded tumor sections from the Grp78 WT and heterozygous mice, and the nuclei were counterstained with DAPI. The upper panel shows the merged image. Tumor sections from the Grp78 WT and heterozygous mice were immunostained with anti-CHOP antibody (lower panel). In all panels, the bars=50 μm.

FIGS. 8A and 8B show the in vitro proliferation and growth rate measurement of tumor cells from the Grp78 WT and heterozygous mice. FIG. 8A shows images of a PCNA staining and immunofluorescent double staining with PCNA and anti-keratin antibody on primary tumor cells from the Grp78 WT and heterozygous mice cultured in vitro for 9 days. In the upper panel, tumor cells from the Grp78 heterozygous mice showed lower PCNA labeling. In the lower panel, immunofluorescent double staining confirmed that the cultured cells were cytokeratin-positive epithelial tumor cells and fewer tumor cells from the Grp78 heterozygous mice showed PCNA labeling. FIG. 8B is a graph showing cell number of tumor cells derived from the Grp78 WT and heterozygous mice (n=7 for each genotype) cultured in vitro. The geometric mean cell number was plotted against time of culture. The 95% CI for the mean cell number on day 9 is (6.9-11.5)×10⁴ for the Grp78+/+, PyT cell lines and (4.9-8.1)×10⁴ for the Grp78+/−, PyT cell lines Analysis of variance showed that the Grp78+/−, PyT cell lines have significantly lower number of cells on day 9 (p=0.048).

FIGS. 9A-D show the analysis of UPR status and caspase activation in tumors from Grp78 WT and heterozygous mice. Lysates of tumors of various sizes were prepared: T1 represents pooled tumors greater than 10 mm in diameter; T2, pooled tumors between 10 and 5 mm in diameter; and T3, pooled tumors smaller than 5 mm in diameter. FIG. 9A is a Western blot with antibodies against GPR78 and β-actin. FIG. 9B is a graph representing relative GRP78 level from the band intensities of the Western blots shown in FIG. 9A. FIG. 9C is a Western blot on the same lysates used in FIG. 9A with antibodies against the respective proteins indicated on the right. HeLa cells treated with 300 nM of thapsigargin (Tg) for 16 hours served as positive control for CHOP induction and ATF6 activation, and the same cells treated with 300 nM of Tg for 6 hours were used as a positive control for eIF2a phosphorylation and activation of ATF4. FIG. 9D is an image showing RT-PCR analysis of XBP-1 mRNA splicing in RNA samples from tumors of different sizes and from NIH3T3 cells either untreated or treated with 300 nM Tg for 16 hours. The positions of the unspliced and spliced forms, as well as the hybrid form of spliced and unspliced XBP-1, are indicated.

FIGS. 10A-D show microvessel density evaluation in tumors and organs from the Grp78 WT and heterozygous mice. Tumors and organs from the Grp78 WT and heterozygous mice were snap-frozen, sectioned, and stained with the endothelial cell specific rat anti-mouse CD31 antibody, and lightly counterstained with H&E. FIG. 10A is an image showing representative tumor section stained with CD31. The bars=100 μm. FIG. 10B is a graph showing tumor microvessel density (MVD) in tumors from the Grp78 WT and heterozygous mice. One unit is defined as one micron for one 200× magnification field. The difference in vessel density is significant (p=0.038). The bars represent the 95% CI for the mean MVD. The results are representative of six animals examined. FIG. 10C shows images of an immunostain of organs with H&E (upper panel) or CD31 (lower panel). The bars=100 μm. FIG. 10D is a graph showing MVD in the indicated organs derived from the Grp78+/+, PyT and Grp78+/−, PyT mice (brain, p=0.25; heart, p=0.85; kidney, p=0.59). The bars represent the 95% CI for the mean MVD.

### DETAILED DESCRIPTION

Tumor vasculature is essential for tumor growth and survival. Despite extensive studies on tumor cells, the expression and function of GRP78 in the tumor vasculature, an integral component of cancer, has not been fully characterized. As described in the examples below, GRP78 is highly elevated in the tumor vasculature, both in situ in tissue and in vitro in primary cell cultures, in contrast to minimal expression in normal tissue. Knockdown of GRP78 by siRNA significantly sensitized blood vessels of malignant glioma tissues (tumor-associate brain endothelial cells; TuBEC) to a variety of chemotherapeutic agents, whereas upregulation of GRP78 in non-malignant brain tissues (brain endothelial cells; BEC) renders them drug resistant. Further, EGCG sensitized TUBEC to chemotherapeutic agents showing small molecules targeting GRP78 enhance the efficacy of chemotherapeutic drugs by eliminating the chemoresistant tumor vasculature.

GRP78 induction occurs during embryonic development and has been widely reported in human cancer. In cancer cell lines, GRP78 promotes survival and chemoresistance in both proliferating and dormant tumor cells. GRP78 has been implicated in proliferation and invasion through activation of the Akt and PAK2 pathways. Autoantibodies against GRP78 in patient sera correlate with aggressive tumor behavior, and retrospective studies revealed that high level GRP78 expression predicts poor survival for cancer patients. Given the importance of GRP78 in cancer cell survival, it is a prime target for discovery of anti-cancer agents. However, because GRP78 controls UPR signaling which has both pro-survival and proapoptotic pathways, downregulation of GRP78 may result in premature activation of the UPR. Therefore, inhibition of GRP78 could be beneficial or harmful to tumor progression, and may affect normal organs and tissues. As described in the examples below, to address this, a Grp78 heterozygous mouse model was used where the basal level and ER stress induction of GRP78 has been determined to be about half of wildtype (WT) level, thus mimicking anti-GRP78 agents that achieve partial suppression of GRP78 expression. In contrast to Grp78 homozygous knockout which results in lethality due to a proliferative defect and massive apoptosis of the inner cell mass of 3.5 day old mouse embryos, the Grp78+/− mice are viable and fertile. Thus, upon breeding with a transgenic mouse model of cancer, the Grp78 heterozygous mice allows examination of the physiological role of GRP78 in in situ generated tumor progression, as compared to normal organ development. As described herein, reduction in GRP78 level by about half in the Grp78 heterozygous mice had no effect on organ development or antibody production, but significantly impeded tumor progression through suppression of proliferation and increase in apoptosis. Further, Grp78 heterozygosity dramatically reduced microvessel density (MVD) in tumors but not in normal organs. Thus, as described herein GRP78 is critical for tumor angiogenesis and has anti-angiogenic activity.

Methods of preventing or reducing tumor angiogenesis in a subject, comprising administering to the subject one or more agents that inhibit expression or activity of GRP78 are provided. Optionally, the method comprises selecting a subject with a tumor in need or prevention or reduction of tumor angiogenesis. Also provided is a method of reducing tumor microvessel density in a subject, comprising selecting a subject with a tumor, wherein the subject is in need or reduction of tumor microvessel density, and administering to the subject one or more agents that inhibit expression or activity of GRP78.

Provided are methods of sensitizing tumor blood vessels to a chemotherapeutic agent comprising administering to the subject one or more agents that inhibit expression or activity of GRP78. Optionally, the method comprises contacting the tumor blood vessels in the subject with one or more agents that inhibit expression or activity of GRP78. Thus, for example, provided is a method of sensitizing tumor blood vessel cells or tumor vasculature to a chemotherapeutic agent in a subject, comprising selecting a subject with a tumor, wherein the cells of the tumor blood vessels or tumor vasculature are resistant to one or more first chemotherapeutic agents, and administering to the subject one or more agents that inhibit expression or activity of GRP78. Optionally, the method further comprises administering one or more second chemotherapeutic agents to the subject. The second chemotherapeutic agent is the same as or different from the one or more first chemotherapeutic agents to which the tumor blood vessel cells or tumor vasculature are resistant.

As used herein, tumor blood vessel cells include, for example, endothelial cells, pericytes and precursors thereof. Blood vessels, including tumor blood vessels, with the exception of capillaries, usually contain three layers. The inner layer contains endothelial cells surrounded by subendothelial connective tissue. The middle layer contains a circularly arranged elastic fiber, connective tissue, and polysaccharide substances. This layer may contain vascular smooth muscle cells, which controls the caliber of the vessel. The outer layer is made of connective tissue. It also contains nerves that supply the muscular layer, as well as nutrient capillaries in the larger blood vessels. Small blood vessels may contain pericytes, also known as Rouget cells or mural cells, which are a mesenchymal-like cells. Capillaries contain a layer of endothelium and occasionally connective tissue.

The compositions and methods provided herein are applicable to the treatment of any tumor having a vascular component. Typical vascularized tumors are solid tumors, particularly carcinomas, which require a vascular component for the provision of oxygen and nutrients. Exemplary solid tumors that may be treated according to the provided methods include, but are not limited to, carcinomas of the lung, breast, ovary, stomach, pancreas, larynx, esophagus, testes, liver, parotid, biliary tract, colon, rectum, cervix, uterus, endometrium, kidney, bladder, prostate, thyroid, squamous cell carcinomas, adenocarcinomas, small cell carcinomas, melanomas, brain (e.g., gliomas and neuroblastomas), and the like. Optionally, the tumor is a glioblastoma including glioblastoma multiforme.

The provided methods comprise administering an agent that reduces or inhibits expression or activity of Grp78. Optionally, the agent is not kringle 5, a derivative of kringle 5 or a variant of kringle 5. Reduction or inhibition of Grp78 can comprise inhibiting or reducing expression of Grp78 mRNA or Grp78 protein, such as by administering antisense molecules, triple helix molecules, ribozymes and/or siRNA. grp78 gene expression can also be reduced by inactivating the grp78 gene or its promoter. The nucleic acids, ribozymes, siRNAs and triple helix molecules for use in the provided methods may be prepared by any method known in the art for synthesis of DNA and RNA molecules. These include techniques for chemically synthesizing oligodeoxyribonucleotides and oligoribonucleotides well known in the art such as for example solid phase phosphoramide chemical synthesis. Alternatively, RNA molecules may be generated by in vitro and in vivo transcription of DNA sequences encoding the nucleic acid molecule. Such DNA sequences may be incorporated into a wide variety of vectors, which incorporate suitable RNA polymerase promoters. Antisense cDNA constructs that synthesize antisense RNA constitutively or inducibly, depending on the promoter used, can be introduced stably into cell lines.

In addition, reduction or inhibition of Grp78 includes inhibiting the activity of the Grp78 protein, referred to herein as Grp78 antagonists. Drugs which target Grp78 have been developed (Ermakova et al., Cancer Res. 66:9260-9 (2006); Davidson et al., Cancer Res. 65:4663-72 (2005); Zhou et al., J. Natl. Cancer Inst. 90:381-88 (1998); Arap et al., Cancer Cell 6:275-84 (2004); Park et al., J. Natl. Cancer Inst. 96:1300-10). Grp78 antagonists also include antibodies, soluble domains of Grp78 and polypeptides that interact with Grp78, such as polypeptides that bind the ATP-binding domain of GRP78, to prevent Grp78 activity. The nucleic acid and amino acid sequence of Grp78 is known in the art. Therefore, variants and fragments of Grp78 that act as Grp78 antagonists can be prepared by any method known to those of skill in the art using routine molecular biology techniques. Numerous agents for modulating expression/activity of intracellular proteins such as GRP in a cell are known. Any of these suitable for the particular system being used may be employed. Typical agents for inhibiting or reducing (e.g., antagonistic) activity of GRPs include mutant/variant GRP polypeptides or fragments and small organic or inorganic molecules.

Inhibitors of Grp78 include inhibitory peptides or polypeptides. As used herein, the term peptide, polypeptide, protein or peptide portion is used broadly herein to mean two or more amino acids linked by a peptide bond. Protein, peptide and polypeptide are also used herein interchangeably to refer to amino acid sequences. The term fragment is used herein to refer to a portion of a full-length polypeptide or protein. It should be recognized that the term polypeptide is not used herein to suggest a particular size or number of amino acids comprising the molecule and that a peptide of the invention can contain up to several amino acid residues or more. Inhibitory peptides include chimeric peptides with Grp78 binding motifs fused to pro-apoptotic sequences (Arap et al., Cancer Cell 6:275-84 (2004), which is incorporated by reference herein in its entirety). Inhibitory proteins also include melanoma differentiation-associated gene-7/interleukin-24 (MDA7/IL-24) and activated form of α-2 macroglobulin (Dent et al., J. Cell Biochem. 95:712-9 (2005); Misra et al., J. Biol. Chem. 281:3694-707 (2006), which are incorporated by reference herein in their entireties).

Inhibitory peptides include dominant negative mutants of a Grp78. Dominant negative mutations (also called antimorphic mutations) have an altered phenotype that acts antagonistically to the wild-type or normal protein. Thus, dominant negative mutants of Grp78 act to inhibit the normal Grp78 protein. Such mutants can be generated, for example, by site directed mutagenesis or random mutagenesis. Proteins with a dominant negative phenotype can be screened for using methods known to those of skill in the art, for example, by phage display.

Nucleic acids that encode the aforementioned peptide sequences are also disclosed. These sequences include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. A wide variety of expression systems may be used to produce peptides as well as fragments, isoforms, and variants. Such peptides or proteins are selected based on their ability to reduce or inhibit expression or activity of Grp78.

Inhibitors of a Grp78 also include, but are not limited to, genistein, (−)-epigallocatechin gallate (EGCG), salicyclic acid from plants, bacterial AB₅ subtilase cytoxin, versipelostatin (Ermakova et al., Cancer Res. 66:9260-9 (2006); Zhou and Lee, J. Natl. Cancer Inst. 90:381-8 (1998); Deng et al., FASEB J. 15:2463-70 (2001); Montecucco and Molinari, Nature 443:511-2 (2006); Park et al., J. Natl. Cancer Inst. 96:1300-10 (2004), which are incorporated herein in their entireties). Inhibitors of GRP78 also include taxanes, such as, for example, paclitaxel and docetaxel in combination with doxirubicin.

Also provided herein are functional nucleic acids that inhibit expression of Grp78. Such functional nucleic acids include but are not limited to antisense molecules, aptamers, ribozymes, triplex forming molecules, RNA interference (RNAi), and external guide sequences. Thus, for example, a small interfering RNA (siRNA) could be used to inhibit expression of Grp78.

Functional nucleic acids are nucleic acid molecules that have a specific function and can interact with a macromolecule. Thus, functional nucleic acids can interact with the mRNA, or genomic DNA. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Representative examples of how to make and use aptamers to bind a variety of different target molecules can be found in, for example, U.S. Pat. Nos. 5,476,766 and 6,051,698.

Ribozymes are nucleic acid molecules that are capable of catalyzing a chemical reaction, either intramolecularly or intermolecularly. There are a number of different types of ribozymes that catalyze nuclease or nucleic acid polymerase type reactions which are based on ribozymes found in natural systems, such as hammerhead ribozymes, hairpin ribozymes and tetrahymena ribozymes). There are also a number of ribozymes that are not found in natural systems, but which have been engineered to catalyze specific reactions de novo (for example, but not limited to U.S. Pat. Nos. 5,807,718, and 5,910,408). Ribozymes may cleave RNA or DNA substrates. Representative examples of how to make and use ribozymes to catalyze a variety of different reactions can be found in U.S. Pat. Nos. 5,837,855; 5,877,022; 5,972,704; 5,989,906; and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. Representative examples of how to make and use triplex forming molecules to bind a variety of different target molecules can be found in U.S. Pat. Nos. 5,650,316; 5,683,874; 5,693,773; 5,834,185; 5,869,246; 5,874,566; and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. Representative examples of how to make and use EGS molecules to facilitate cleavage of a variety of different target molecules can be found in U.S. Pat. Nos. 5,168,053; 5,624,824; 5,683,873; 5,728,521; 5,869,248; and 5,877,162.

Gene expression can also be effectively silenced in a highly specific manner through RNA interference (RNAi). Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. Sequence specific gene silencing can be achieved in mammalian cells using synthetic, short double-stranded RNAs that mimic the siRNAs produced by the enzyme dicer. siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit (Ambion, Austin, Tex.).

Proteins that inhibit Grp78 include antibodies with antagonistic or inhibitory properties. Antibodies to Grp78 are commercially available, for example, from Santa Cruz Biotechnology (Santa Cruz, Calif.). In addition to intact immunoglobulin molecules, fragments, chimeras, or polymers of immunoglobulin molecules are also useful in the methods taught herein, as long as they are chosen for their ability to inhibit Grp78. The antibodies can be tested for their desired activity using in vitro assays, or by analogous methods, after which their in vivo therapeutic or prophylactic activities are tested according to known clinical testing methods.

The term antibody is used herein in a broad sense and includes both polyclonal, single-chain and monoclonal antibodies. Functional fragments thereof are also useful in the provided methods. Monoclonal antibodies can be made using any procedure that produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro. The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

Digestion of antibodies to produce fragments thereof, e.g., Fab fragments, can be accomplished using routine techniques known in the art. For instance, digestion can be performed using papain. Examples of papain digestion are described in WO 94/29348 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross linking antigen.

The antibody or fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curt Opin. Biotechnol. 3:348-354, 1992).

As used herein, the term antibody or antibodies can also refer to a human antibody and/or a humanized antibody. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1):86 95, 1991). Human antibodies (and fragments thereof) can also be produced using phage display libraries (Hoogenboom et al., J. Mol. Biol., 227:381, 1991; Marks et al., J. Mol. Biol., 222:581, 1991). The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551 255 (1993); Jakobovits et al., Nature, 362:255 258 (1993); Bruggermann et al., Year in Immunol., 7:33 (1993)). Specifically, the homozygous deletion of the antibody heavy chain joining region (J(H)) gene in these chimeric and germ line mutant mice results in complete inhibition of endogenous antibody production, and the successful transfer of the human germ line antibody gene array into such germ line mutant mice results in the production of human antibodies upon antigen challenge.

Antibody humanization techniques generally involve the use of recombinant DNA technology to manipulate the DNA sequence encoding one or more polypeptide chains of an antibody molecule. Accordingly, a humanized form of a non human antibody (or a fragment thereof) is a chimeric antibody or antibody chain that contains a portion of an antigen binding site from a non-human (donor) antibody integrated into the framework of a human (recipient) antibody. Fragments of humanized antibodies are also useful in the methods taught herein. As used throughout, antibody fragments include Fv, Fab, Fab′, or other antigen binding portion of an antibody. Methods for humanizing non human antibodies are well known in the art. For example, humanized antibodies can be generated according to the methods of Winter and co workers (Jones et al., Nature, 321:522 525 (1986), Riechmann et al., Nature, 332:323 327 (1988), Verhoeyen et al., Science, 239:1534 1536 (1988)), by substituting rodent CDRs or CDR sequences for the corresponding sequences of a human antibody. Methods that can be used to produce humanized antibodies are also described in U.S. Pat. No. 4,816,567 (Cabilly et al.); U.S. Pat. No. 5,565,332 (Hoogenboom et al.); U.S. Pat. No. 5,721,367 (Kay et al.); U.S. Pat. No. 5,837,243 (Deo et al.); U.S. Pat. No. 5,939,598 (Kucherlapati et al.); U.S. Pat. No. 6,130,364 (Jakobovits et al.); and U.S. Pat. No. 6,180,377 (Morgan et al.).

The compositions and agents that reduce or inhibit Grp78 are optionally administered in vivo in a pharmaceutically acceptable carrier. By pharmaceutically acceptable is meant a material that is not biologically or otherwise undesirable. Thus, the material may be administered to a subject, without causing undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

In the provided methods, the agent or composition is administered in a manner so that it can ultimately contact the target tumor blood vessels or tumor blood vessel cells, for example, systemically. The route by which the agent or composition is administered, as well as the formulation, carrier or vehicle, depends on the location as well as the type of the target cells. A wide variety of administration routes can be employed. For example, for a solid tumor that is accessible, the agent or composition can be administered by injection directly to the tumor. Alternatively, for example, the agent or composition can be administered intravenously or intravascularly. The agent or composition can also be administered subcutaneously, intraperitoneally, intrathecally (e.g., for brain tumor), topically (e.g., for melanoma), orally (e.g., for oral or esophageal cancer), rectally (e.g., for colorectal cancer), vaginally (e.g., for cervical or vaginal cancer), nasally, by inhalation spray or by aerosol formulation (e.g., for lung cancer).

Suitable carriers and their formulations are described in Remington: The Science and Practice of Pharmacy (21st ed.) eds. A. R. Gennaro et al., University of the Sciences in Philadelphia 2005. Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like. Examples of the pharmaceutically-acceptable carriers include, but are not limited to, saline, Ringer's solution and dextrose solution. The pH of the solution is preferably from about 5 to about 8.5, and more preferably from about 7.8 to about 8.2. Further carriers include sustained release preparations such as semipermeable matrices of solid hydrophobic polymers containing the antibody, which matrices are in the form of shaped articles, e.g., films, liposomes or microparticles. Certain carriers may be more preferable depending upon, for instance, the route of administration and concentration of composition being administered. Pharmaceutical carriers are known may be standard carriers for administration of drugs to humans, including solutions such as sterile water, saline, and buffered solutions at physiological pH. Other compounds will be administered according to standard procedures used by those skilled in the art.

The terms effective amount and effective dosage are used interchangeably. The term effective amount is defined as any amount necessary to produce a desired physiologic response. Effective amounts and schedules for administering the compositions may be determined empirically, and making such determinations is within the skill in the art. The dosage ranges for the administration of the compositions are those large enough to produce the desired effect in which the symptoms or disorder are affected. The dosage should not be so large as to cause substantial adverse side effects, such as unwanted cross-reactions, anaphylactic reactions, and the like. The dosage of the agent or composition required will vary from subject to subject, depending on the species, age, weight and general condition of the subject, the severity of the airway disorder being treated, the particular active agent used, its mode of administration and the like. Thus, it is not possible to specify an exact amount for every composition. However, an appropriate amount can be determined by one of ordinary skill in the art using only routine experimentation given the teachings herein.

The provided compositions can be administered in combination with one or more other therapeutic or prophylactic regimens. As used throughout, a therapeutic agent is a compound or composition effective in ameliorating a pathological condition. Illustrative examples of therapeutic agents include, but are not limited to, an anti-cancer compound, anti-inflammatory agents, anti-viral agents, anti-retroviral agents, anti-opportunistic agents, antibiotics, immunosuppressive agents, immunoglobulins, and antimalarial agents.

An anti-cancer compound or chemotherapeutic agent is a compound or composition effective in inhibiting or arresting the growth of an abnormally growing cell. Thus, such an agent may be used therapeutically to treat cancer as well as other diseases marked by abnormal cell growth. A pharmaceutically effective amount of an anti-cancer compound is an amount administered to an individual sufficient to cause inhibition or arrest of the growth of an abnormally growing cell. Illustrative examples of anti-cancer compounds include: CPT-11, temozolomide (TMZ), bleomycin, carboplatin, chlorambucil, cisplatin, colchicine, cyclophosphamide, daunorubicin, dactinomycin, diethylstilbestrol doxorubicin, etoposide, 5-fluorouracil, floxuridine, melphalan, methotrexate, mitomycin, 6-mercaptopurine, teniposide, 6-thioguanine, vincristine and vinblastine.

Any of the aforementioned treatments can be used in any combination with the compositions described herein. Thus, for example, the compositions can be administered in combination with a chemotherapeutic agent and radiation. Other combinations can be administered as desired by those of skill in the art. Combinations may be administered either concomitantly (e.g., as an admixture), separately but simultaneously (e.g., via separate intravenous lines into the same subject), or sequentially (e.g., one of the compounds or agents is given first followed by the second). Thus, the term combination is used to refer to either concomitant, simultaneous, or sequential administration of two or more agents.

As used throughout, by a subject is meant an individual. Thus, the subject can include domesticated animals, such as cats, dogs, etc., livestock (e.g., cattle, horses, pigs, sheep, goats, etc.), laboratory animals (e.g., mouse, rabbit, rat, guinea pig, etc.) and birds. Preferably, the subject is a mammal such as a primate including a human.

As used herein, references to decreasing, reducing, or inhibiting include a change of 10, 20, 30, 40, 50, 60, 70, 80, 90 percent or greater as compared to a control level. Such terms can include but do not necessarily include complete elimination.

As used herein the terms treatment, treat or treating refers to a method of delaying or reducing the effects of a disease or condition or symptom of the disease or condition. Thus in the disclosed method treatment can refer to a 10%, 20%, 30%, 40%, 50%, 60%, 70%, 80%, 90% or 100% reduction in the severity of an established disease or condition or symptom of the disease or condition. For example, a method for treating a disease is considered to be a treatment if there is a 10% reduction in one or more symptoms or clinical characteristics (e.g., tumor size) of the disease in a subject as compared to control. Thus the reduction can be a 10, 20, 30, 40, 50, 60, 70, 80, 90, 100% or any percent reduction in between 10 and 100 as compared to native or control levels. It is understood that treatment does not necessarily refer to a cure or complete ablation of the disease, condition or symptoms of the disease or condition.

As used herein, the terms prevent, preventing and prevention of a disease or disorder refers to an action, for example, administration of a therapeutic agent, that occurs before a subject begins to suffer from one or more symptoms of the disease or disorder, which inhibits or delays onset of the severity of one or more symptoms of the disease or disorder.

There are a variety of sequences related to, for example, Grp78 that are disclosed on Genbank, at www.pubmed.gov, and these sequences and others are herein incorporated by reference in their entireties as well as for individual subsequences contained therein.

Disclosed are materials, compositions, and components that can be used for, can be used in conjunction with, can be used in preparation of, or are products of the disclosed methods and compositions. These and other materials are disclosed herein, and it is understood that when combinations, subsets, interactions, groups, etc. of these materials are disclosed that, while specific reference to each various individual and collective combinations and permutation of these compounds may not be explicitly disclosed, each is specifically contemplated and described herein. For example, if a method is disclosed and discussed and a number of modifications can be made to materials used in the method or in the steps of the method, each and every combination and permutation of the method and the modifications that are possible are specifically contemplated unless specifically indicated to the contrary. Likewise, any subset or combination of these is also specifically contemplated and disclosed. Thus, if there is a variety of additional steps that can be performed in a method, it is understood that each of these additional steps can be performed with any specific method steps or combination of method steps of the disclosed methods, and that each such combination or subset of combinations is specifically contemplated and should be considered disclosed. Thus, a number of aspects have been described. Nevertheless, it will be understood that various modifications may be made. Furthermore, when one characteristic or step is described, it can be combined with any other characteristic or step herein even if the combination is not explicitly stated. Accordingly, all combination of disclosed agents, steps and characteristics are provided even in the absence of explicit disclosure herein.

Throughout this application, various publications are referenced. The disclosures of these publications in their entireties are hereby incorporated by reference into this application in order to more fully describe the state of the art to which this invention pertains.

A number of aspects have been described. Nevertheless, it will be understood that various modifications may be made. Furthermore, when one characteristic or step is described it can be combined with any other characteristic or step herein even if the combination is not explicitly stated. Accordingly, other aspects are within the scope of the claims.

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention except as and to the extent that they are included in the accompanying claims. Efforts have been made to ensure accuracy with respect to numbers (e.g., amounts, temperature, etc.), but some errors and deviations should be accounted for.

### EXAMPLES

### Example 1

### Grp78 Sensitizes the Tumor Vasculature to Chemotherapeutic Drugs

### Materials and Methods

Cell Culture. Endothelial cells (EC) were isolated from normal human brain tissue or human glioma tissue as previously described (Charalambous et al, J. Neurosurg., 102:699-705, 2005; Charalambous et al, Exp Cell Res., 313:1192-202, 2007). All experiments were performed on subconfluent (60-80%) cultures, in passages 4 to 6 only.

EC were cultured in RPMI 1640 medium (GIBCO Laboratories; Grand Island, N.Y.) supplemented with 100 ng/ml endothelial cell growth supplement (Upstate Biotechnologies; Rochester, N.Y.), 2 mM L-Glutamine (GIBCO), 10 mM Hepes (GIBCO), 24 mM sodium bicarbonate (GIBCO), 300 units heparin USP (Sigma-Aldrich; St. Louis, Mo.), 1% penicillin/streptomycin (GIBCO) and 10% fetal calf serum (FCS) (Omega Scientific; Tarzana, Calif.). Purity was analyzed by immunostaining for the following EC markers: CD31/PECAM-1 (Santa Cruz Biotechnology; Santa Cruz, Calif.), von Willebrand Factor (DAKO; Carpinteria, Calif.), and CD105/endoglin (Santa Cruz Biotechnology). Cultures were greater than 98% positive for these markers and negative for the astrocyte/glioma marker, glial fibrillary acidic protein (DAKO), and the macrophage/microglia marker, CD11b (Immunotech; Villepinte, France).

MTT cell viability assay. Cells were plated in triplicate (3×10³ cells/well; 100 ml/well) into 96-well plates coated with 1% gelatin. The 3-(4,5-dimethylthiazol-2-yl)-2,5-diphenyltetrazolium bromide (MTT) assay was performed according to the manufacturer's instructions (Sigma-Aldrich, St. Louis, Mo.). Percent cell viability was calculated relative to vehicle-treated controls.

Cell death ELISA. EC were treated with etoposide (Calbiochem; La Jolla, Calif.), temozolomide (TMZ) (Schering-Plough; Kenilworth, N.J.), CPT-11 (Pharmacia; New York, N.Y.), the caspase inhibitor Q-VDOPH (Calbiochem), or EGCG (Sigma-Aldrich, St. Louis, Mo.), then assayed using the Cell Death Detection ELISA Plus kit (Roche Diagnostics; Indianapolis, Ind.) according to manufacturer's protocol. Percent death was calculated based on 100% positive cell death control.

Lentiviral construct. The sequences of siRNA, the isolation of human Grp78 cDNA and their subcloning into lentiviral constructs are previously described (Dong et al, Cancer Res., 65:5785-91, 2005). The sequences of the siRNA against human GRP78 are: siGRP78A: 5′-GGAGCGCAUUGAUACUAGATT-3′ (SEQ ID NO:1) and siGRP78B: 5′-AAGAAAAGCUGGGAGGUAAAC-3′ (SEQ ID NO:2). The sequences of the control siRNA are: sicontrol A: 5′-GGAGAAGAAUAGCAACGGUAA-3′ (SEQ ID NO:3); sicontrol B: 5′-AAGGUGGUUGUUUUGUUCATT-3′ (SEQ ID NO:4). For construction of lentivirus expressing GRP78, full length human GRP78 was prepared by reverse transcription of total HEK293T RNA followed by a 2-step PCR amplification and subcloning into the BamHI/XhoI sites of pcDNA3 (Invitrogen; Carlsbad, Calif.) to yield pcDNA3-hGRP78. Non-replicating lentiviral vectors co-expressing bicistronic human GRP78 and EGFP linked via the EMCV IRES were produced using the pLenti6/V5-D-TOPO and VIRAPOWER™ Lentivirus Expression system (Invitrogen, Carlsbad, Calif.). Initially, EGFP (Clonetech; Mountain View, Calif.) was inserted into the CMV driven expression cassette by TOPO cloning. After the viability of this construct was established, the parent construct was modified. The human GRP78 gene digested from pcDNA3-hGRP78 using XbaI and XhoI was ligated into pLenti6/EGFP between the CMV promoter and the EGFP gene. Next, an insert encoding the EMCV IRES, was generated by PCR from pIRES-EGFP (Clonetech) and subcloned into the XhoI/AgeI sites of pLenti6/huGRP78 EGFP. The IRES sequence was inserted between the human GRP78 gene and the EGFP allowing hGRP78 expression to be monitored by EGFP fluorescence. The manufacturer's manual was followed for TOPO cloning and production of viral particles. For infection, 10⁴ cells were plated in 6-well dishes and infected with lentivirus at titers of 5×10⁶ units/ml (TU/ml). Infected cells, monitored for GFP, were used when cultures were 100% GFP positive.

Immunostaining. Cytocentrifuge cell preparations and cryostat tissue sections were stained using anti-GRP78 polyclonal antibody as published (Charalambous et al, J. Neurosurg., 102:699-705, 2005). Cytocentrifuge cell preparations and cryostat tissue sections were acetone fixed, stained with the primary antibody, anti-GRP78 polyclonal antibody (1:100) (Santa Cruz Biotechnology, Santa Cruz, Calif.), followed by the secondary biotinylated goat anti-rabbit antibody (1:400) (Vector Laboratories, Burlingame, Calif.). Subsequently the slides were treated with the avidin-biotin peroxidase complex (Vector Laboratories), followed by the aminoethyl carbazol (AEC) substrate kit (Vector Laboratories). Hematoxylin was the nuclear counterstain. For double immunofluorescence, tissues were incubated with both polyclonal anti-GRP78 and monoclonal anti-CD31 antibodies, and subsequently stained with Texas-red labeled anti-rabbit antibody and fluorescein-green labeled anti-mouse antibody. Nuclei were labeled using DAPI mounting medium. Staining controls included isotype matched serum.

Western blots. Protocols for Western blot analysis have been described (Mao et al, Nat. Med., 10:1013-4, 2004). EC were lysed, proteins were electrotransferred onto nitrocellulose membranes, and probed with anti-GRP78, anti-b-actin (Santa Cruz Biotechnology, Santa Cruz, Calif.) or anti-GAPDH (Santa Cruz Biotechnology) polyclonal antibodies. Protein bands were detected by chemiluminescence using the SUPERSIGNAL™ substrate (Pierce Biotechnology, Rockford, Ill.) and analyzed using a Phosphoimager (Hope Micro-Max, Freedom Imaging; Anaheim, Calif.).

Statistical analysis. The values presented were calculated as mean and SEM. Statistical significance was evaluated using the Student's two-tailed t-test, with p<0.05 considered significant.

### Results

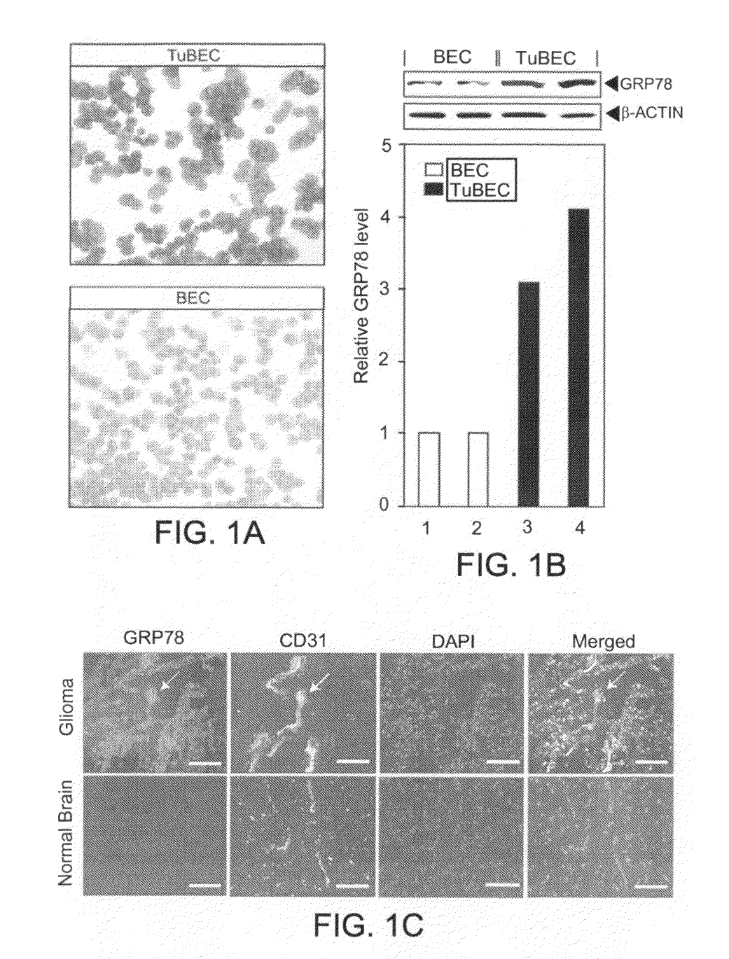

Glucose-regulated protein 78 (GRP78) expression is highly elevated in human tumor-associated brain endothelial cells. To study the functional properties of tumor vasculature, purified human primary Tumor Brain Endothelial Cells (TuBEC) and control Brain Endothelial Cells (BEC) were used. To test whether TuBEC expressed GRP78, subconfluent cultures of five different TuBEC patient samples and four different BEC specimens were immunostained with anti-GRP78. Results demonstrated that TuBEC were more positive for GRP78 expression than BEC (FIGS. 1A and 1B). To quantitate differences in GRP78 levels, Western blots were performed, and examples of TuBEC specimens from two patients and BEC specimens from two patients were shown (FIG. 1B). The results demonstrate that GRP78 expression in TuBEC is 3- to 4-fold higher compared to BEC.

To confirm this observation in the tumor vasculature in tissues in situ, frozen sections of glioma were immunostained for either GRP78 or the endothelial cell marker CD31, with DAPI staining the nuclei (FIG. 1C). GRP78 was expressed in both the tumor vasculature and glioma cells. By contrast, normal brain tissues exhibited CD31 staining vessels, but minimal staining for GRP78 (FIG. 1C). Merged images confirmed minimal GRP78 expression in normal brain and vasculature.

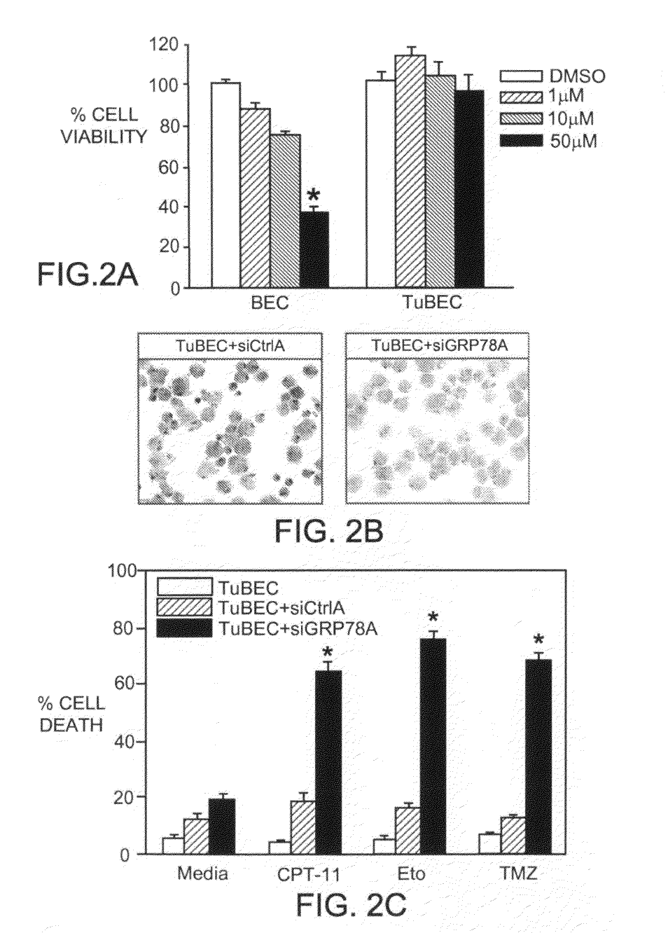

GRP78 regulates chemoresistance in TuBEC. To compare the sensitivities of TuBEC and BEC to chemotherapeutic agents, cells were treated with the topoisomerase II inhibitor, etoposide [1-50 μM] or DMSO (vehicle) [0.1%] for 72 hours, and cytoxicity was assessed using the MTT assay, which was performed according to manufacturer's protocol (Sigma Aldrich; St. Louis, Mo.). At 50 μM etoposide, over 60% of BEC died, whereas no significant cell death was observed with TuBEC (FIG. 2A).

To determine the effects of reducing GRP78 expression, TuBEC were infected with lentivirus expressing control siRNA (siCtr1A) or siRNA specifically targeted against human GRP78 (siGRP78A). Five days post-infection, cell preparations were analyzed for GRP78 protein using immunostaining. GRP78 protein was reduced by siGRP78A at 14 days post-infection (FIG. 2B); no change was observed with siCtr1A as compared with uninfected TuBEC (FIG. 1A). GRP78 protein levels remained low for at least 3 passages, approximately three weeks.

To test whether GRP78 confers drug resistance to TuBEC, TuBEC cultures were infected with lentivirus expressing siGRP78A and siCtr1A. Five days post infection, cultures were left untreated (media), or treated with CPT-11 [100 μM], etoposide (Eto) [50 μM] or temozolomide (MZ) [300 μM]. After treatment for seven days, the cells were analyzed for cytotoxicity. Untreated cells were resistant to these drugs (<10% cell death); however, TuBEC infected with siGRP78A exhibited an increase (p<0.01) in cytotoxicity with each drug tested (FIG. 2C). Infection with siGRP78A alone, without drug treatment, did not increase cell death as compared to infection with control siRNA (FIG. 2C).

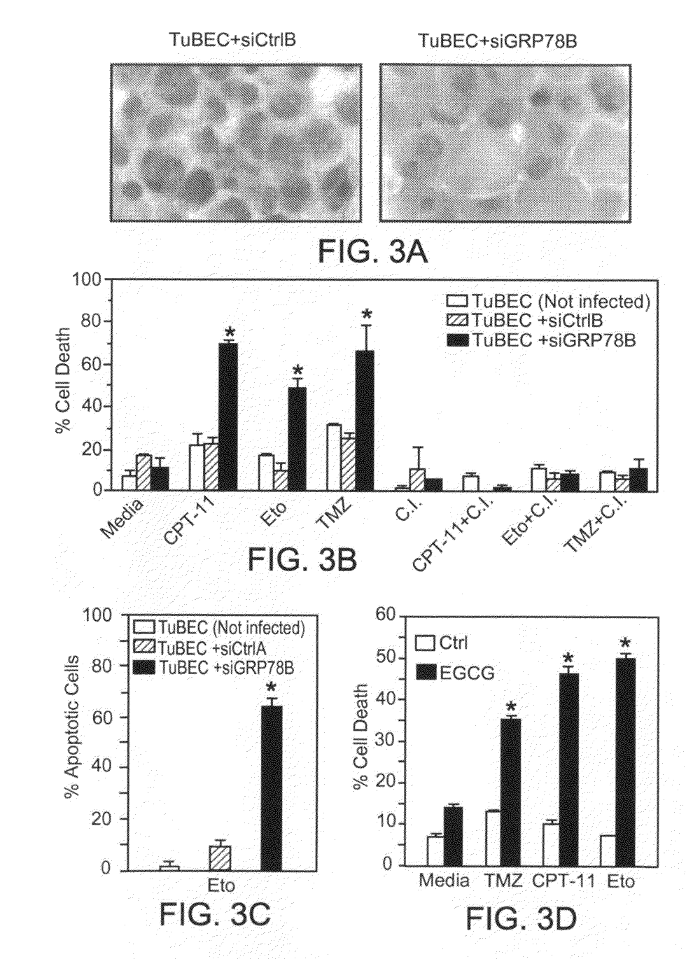

GRP78 siRNA induces caspase-dependent cell death in TuBEC. To eliminate potential non-specific effects of siRNA to GRP78, cytotoxicity measurements were confirmed in TuBEC infected with a second siRNA targeted against human GRP78 (siGRP78B); another control siRNA (siCtr1B) was also used. The ability of siGRP78B to suppress GRP78 expression in TuBEC was confirmed by immunostaining (FIG. 3A); siCtr1B did not reduce staining, as was observed previously with siCtr1A (FIG. 2A).

To determine whether the observed cell death induced by reduced GRP78 was caspase-dependent, cells were treated with CPT-11, Eto, or TMZ in the absence or presence of the caspase inhibitor Q-VDOPH [10 mM] for seven days. Caspase inhibition blocked cell death with all three drugs (FIG. 3B).

To confirm that GRP78 knockdown induced apoptotic cell death, the TUNEL assay was performed with uninfected TuBEC or TuBEC infected with lentivirus siGRP78B or siCtr1A, and treated with Eto [50 μM] (FIG. 3C). Cells infected with siGRP78B show an increase in apoptosis compared to untreated and siCtr1A infected cells (FIG. 3C).

Inhibition of GRP78 activity by EGCG enhances chemosensitivity in TuBEC. To target GRP78 through a small molecule approach, TuBEC were incubated with EGCG alone or in combination with TMZ, CPT-11, and Eto. Cytotoxicity was measured after seven days (FIG. 3D). Treatment with TMZ, CPT-11, Eto or EGCG alone did not cause TuBEC cell death; however, combing EGCG with TMZ (35% cell death, p=0.003), CPT-11 (46% cell death, p=0.005), or Eto (49% cell death, p=0.001) caused increased cell death (FIG. 3D).

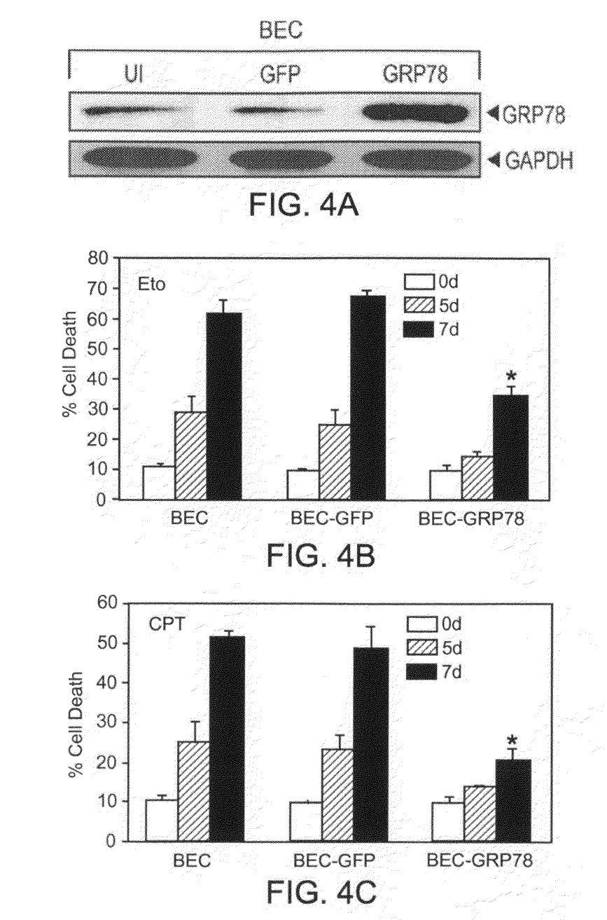

Overexpression of GRP78 causes drug resistance in normal endothelial cells. To determine whether GRP78 overexpression is a key contributing factor to drug resistance in endothelial cells, BEC, which express low levels of GRP78 and are sensitive to therapeutic agents, were left uninfected or infected with lentivirus expressing either green fluorescent protein (GFP) or GRP78. After five days, BEC infected with GRP78 exhibited overexpression of GRP78 (FIG. 4A). BEC were then treated with Eto [50 μM] or CPT-11 [100 μM] for five or seven days and were tested for cytotoxicity. After seven days, GRP78 overexpression in BEC provided protection against both Eto and CPT-11 (FIG. 4B and FIG. 4C).

### Example 2

### Role of GRP78 in Tumor Proliferation, Survival, and Angiogenesis

### Materials and Methods

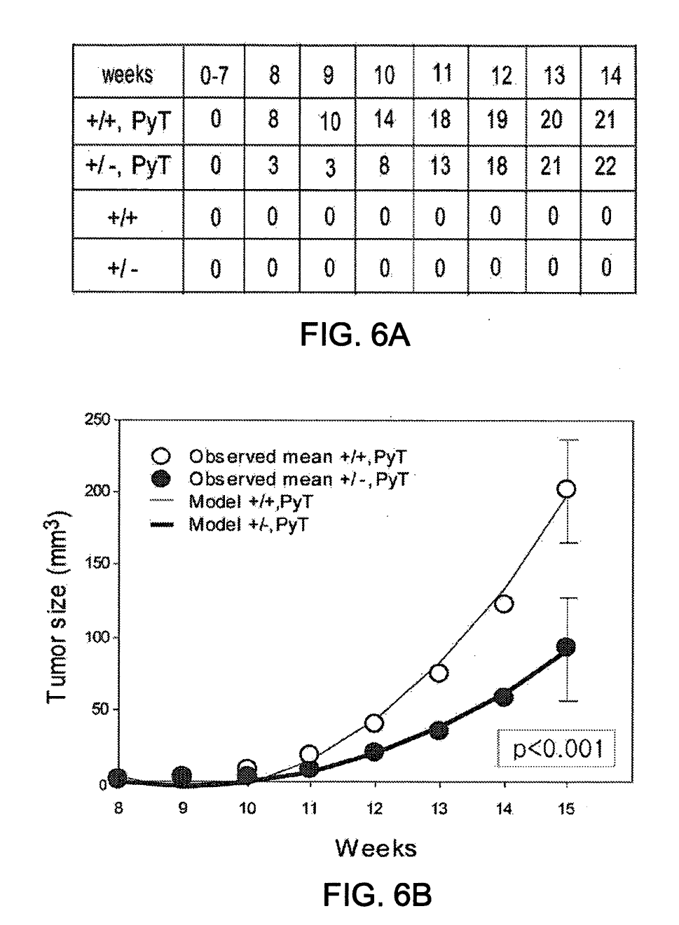

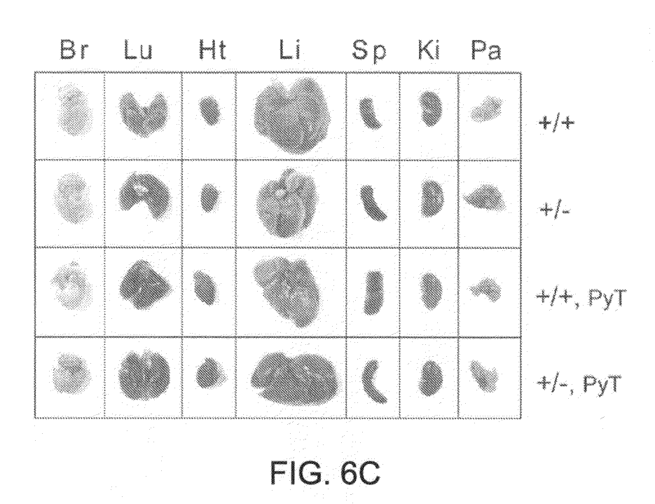

Generation of mammary tumors in genetically altered Grp78 mice and monitoring of tumor growth. The generation of the Grp78+/− mice has been described (Luo et al, Mol. Cell. Bio., 26:5688-97, 2006). Female Grp78+/− mice were mated with male MMTVPyVT heterozygous transgenic mice (Guy et al, Mol. Cell. Bio., 12:954-61, 1992). The progenies were genotyped by PCR of tail DNA and monitored for tumor growth and incidence. The experiment on tumor growth was done in two phases. Seven Grp78+/+, PyT mice and six Grp78+/−, PyT mice were used in the initial phase. Starting 8 weeks of age, mammary tumors were detected. The primary tumor diameters were measured with a caliper weekly. The second phase was a replication of the first one, with two modifications: 1) The sample size in phase 2 was 15 mice in each group, and 2) both the width and the length of the tumor were measured in phase 2. Tumor volume was calculated using the equation: Volume=Width²×Length×0.5. Following sacrifice at the end of the experiment, organs and tumors were harvested for analysis. The numbers of mice with tumor at each week from both phases are shown in FIG. 6A and the tumor growth curves in the second phase are shown in FIG. 6B.

Immunization and determination of immunoglobulin titers. Six to 8 week old Grp78+/− and littermate Grp78+/+ mice were immunized by intraperitoneal injections of 100 pg trinitrophenyl-keyhole limpet hemocyanin (TNP-KLH, Biosearch Technologies; Novato, Calif.) in Imject alum (Pierce Biotechnology; Rockford, Ill.). TNP-KLH-immunized mice were given a booster 21 days later. Serum samples were collected before immunization and on day 7 after TNP-KLH re-immunization. Basal immunoglobulin levels in sera were quantified using BEADLYTE® mouse immunoglobulin isotyping kit (Millipore; Billerica, Mass.) following the manufacturer's protocol. To measure relative TNP-specific antibody levels, the same isotyping kit combined with BEADLYTE® biotin-conjugated trinitrophenyl ovalbumin (TNP-OVA, Millipore; Billerica, Mass.) and BEADLYTE® streptavidin-phycoethrin (Millipore; Billerica, Mass.) were used following the manufacturer's protocol.

Derivation of cell culture from tumor tissues. Primary culture of tumor cells was established as described (Dubeau et al, Cancer Res, 47:2107-12, 1987). Briefly, tumor tissues were cut into fine pieces and seeded onto 6 cm diameter culture dishes and cultured at 37° C. with 5% CO₂ in 1 ml of high glucose Dulbecco's Modified Eagle's Medium (DMEM) containing 4.5 mg/ml glucose supplemented with 10% fetal bovine serum, 2 mM glutamine, and 1% penicillin-streptomycin-neomycin antibiotics. After overnight culturing, an additional 2 nil of medium was added. After 5 days, the tumor tissues were removed from the culture dish, and the attached cells were allowed to grow. After expansion, the cells were seeded for immunohistochemical (IHC) staining and growth rate measurements. For growth measurements, the cells were stained with Trypan Blue and counted every two days until day 9.

Immunohistochemical and immunofluorescent staining. IHC staining was performed on paraffin-embedded tumor sections (4 pm) or cell culture in a chamber slide (Nalge Nunc International; Naperville, Ill.). Vectastain elite ABC kit (Vector Lab; Burlingame, Calif.) was used for immunohistochemistry, and fluorescein or Texas red conjugated anti-rabbit or mouse IgG (H+L) (Vector Lab) for immunofluorescent staining. Primary antibodies against Pan-cytokeratin (rabbit, 1:50), GRP78 (rabbit, 1:100), CHOP (mouse, 1:200) and PCNA (mouse, 1:50) were from Santa Cruz Biotechnology (Santa Cruz, Calif.). Antibody against human vimentin (mouse, 1:100) was from Chemicon International (Temecula, Calif.). ProLong gold anti-fade mounting medium with DAPI (Invitrogen, Carlsbad, Calif.) and aqueous mounting medium were from Vector Lab.

IHC staining was carried out as described previously (Dong et al, Cancer Res., 65:5785-91, 2005). Immunofluorescent staining of cultured cells was performed on cells plated in a chamber slide. The cells were fixed with methanol for 30 min at −20° C. Three washes with PBS were followed by permeabilization with 0.1% Triton X-100 in PBS (v/v) for 10 minutes at room temperature and blocking for 60 minutes at room temperature with 1% bovine serum albumin in PBS (w/v). The treated cells were incubated with the first primary antibody at 4° C. overnight in a humidified chamber. After washing with PBS three times, secondary antibody conjugated with fluorescein or Texas red was added at a final concentration of 10 μg/ml and incubated for 60 minutes at room temperature. For immunofluorescent double staining, the cells were washed with PBS three times and the above staining procedure was repeated for the second primary and second secondary antibodies, respectively. The stained cells were mounted with aqueous mounting medium or ProLong gold anti-fade mounting medium with DAPI, the processed cells were visualized using a fluorescence microscope.

TUNEL assay. In Situ Cell Death Detection Kit, TMR red (Roche Applied Science, Indianapolis, Ind.) was used and the sections of paraffin-embedded tumor were stained following the protocol provided by the manufacturer. The tumor sections were mounted with ProLong gold anti-fade mounting medium with DAPI. The apoptotic cells were visualized using a fluorescence microscope.

Microvessel density measurement. Frozen mouse tissues (tumor, kidneys, heart, and brain) were sectioned at 5 μm and fixed with acetone and tissue sections were stained overnight with rat anti-mouse CD31 antibody (BD Pharmingen; San Jose, Calif.) followed by biotinylated anti-rat antibody (Vector Lab; Burlingame, Calif.) for 45 minutes as previously described (Hofinan et al, Blood, 92:3064-72, 1998). Tissues were then treated with avidin-biotin peroxidase complex (Vector Lab) for 30 minutes and the aminoethyl carbazol (AEC) substrate kit for 10 minutes (Vector Lab). Counterstaining was performed with hematoxylin. Isotype-matched serum was used in place of the primary antibody for staining controls. Positive staining was quantified using the imaging processing program ImageJ (National Institute of Health; http://rsbweb.nih.gov/ij/). Tumor microvessel density (MVD) staining was analyzed in 6 mice, 3 from each genotype and the evaluation was performed on 5 random fields per tumor. Organ MVD staining was analyzed in a pair of Grp78 WT and heterozygous mice from the same group, with five random fields examined per tissue.

Western blots. Pieces of tumors dissected from mice were homogenized with a Dounce homogenizer in radioimmunoprecipitation assay buffer (50 mM Tris, pH 8, 150 mM NaCl, 1% NP-40, 0.5% deoxy cholate, 0.1% SDS, 10% glycerol, and protease inhibitors) to lyse cells, followed by centrifugation at 4° C. for 10 minutes. The supernatant obtained from each sample was subjected to immunoblot. The primary antibodies used were: rabbit anti-GRP78 (1:1000), mouse anti-CHOP (1:1000), rabbit anti-ATF6 (1:1000) and rabbit anti-ATF4 (1:1000) from Santa Cruz Biotechnology (Santa Cruz, Calif.), mouse anti-p-actin (1:5000) from Sigma-Aldrich (St. Louis, Mo.), mouse anti-caspase-7 (1:4000) from BD Pharmingen (San Jose, Calif.), rabbit anti-cleaved caspase-3 (1:1000) and anti-p-eIF2a and rabbit anti-eIF2a (1:1000) from Cell Signaling (Danvers, Mass.). The experiments were repeated 2 to 3 times.

XBP-1 splicing measurement. Total RNA from tumor samples were isolated using Trizol reagent (Invitrogen; Carlsbad, Calif.), and first strand cDNA was synthesized with Superscript II (Invitrogen). To detect both spliced and =spliced forms of XBP-1, nested PCR was performed. The primers used for first round PCR were: 5′-TAG AAA GAAAGCCCGGATGA-3′ (SEQ ID NO:5) (forward) and 5′-AAAGGGAGGCTGGTAAGGAA-3′ (SEQ ID NO:6) (reverse). PCR primers for the second round were: 5′-GAACCAGGAGTTAAGAACACG-3′ (SEQ ID NO:7) (forward) and 5′-AGGCAACAGTGTCAGAGTCC-3′ (SEQ ID NO:8) (reverse).

Statistical Analysis. The tumor volumes of the Grp78 WT and heterozygous mice were compared using the random coefficient model where the intercept and the slope for each mouse were treated as random. A quadratic time term was included in the model because of the curvature of the growth pattern. The group x time interaction was tested using the likelihood ratio test, which will indicate whether the growth pattern was significantly different between the two groups. The growth rates of multiple primary tumor cell lines derived from Grp78 WT and heterozygous mice were monitored in two experiments. Prior to analysis, the cell numbers were normalized so that all cell lines started at the same level with the number of cells being 2×10⁴ and log-transformed Analysis of variance (ANOVA) was performed to compare the cell numbers on day 9 between the cell lines from Grp78 WT and heterozygous tumors after adjusting for the experiment. The microvessel density in tumors and organs of heterozygous mice were also compared to that of WT mice using ANOVA after log-transforming the data. The means and 95% CI were calculated on the log transformed data and then transformed back to the original scale.

### Results

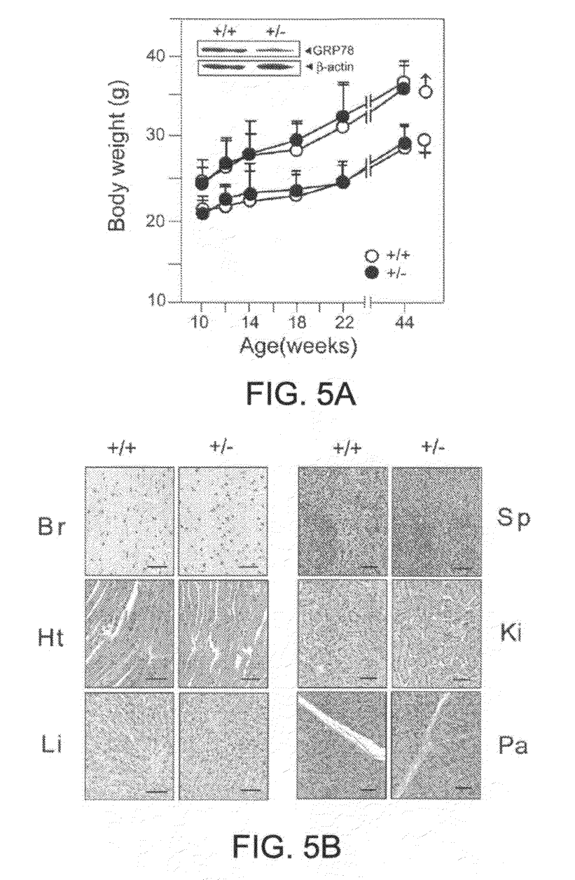

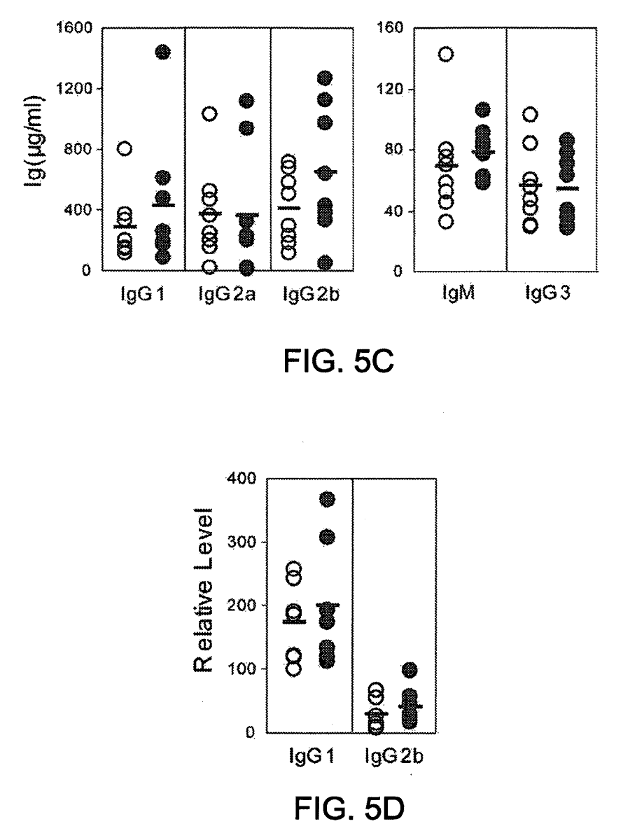

Grp78 heterozygous mice exhibit normal growth, organ development and antibody production. Both male and female Grp78+/− mice grew at the same rate as their WT siblings (FIG. 5A) and the size, morphology and histology of their major organs were comparable to those of WT (FIG. 5B). As the immunoglobulin (Ig) binding protein, GRP78 is highly abundant in plasma cells where it stabilizes Ig chains and facilitates their assembly (Hendershot, LM, Mt. Sinai J Med, 71:289-97, 2004). Analysis of pre-immune serum IgM and various subclasses of IgG antibody levels yielded no significant difference between the WT and heterozygous mice (FIG. 5C). After immunization with the antigen TNP-KLH, the relative serum levels of IgG1 and IgG2b specific to TNP remained similar (FIG. 5D).

Grp78 heterozygosity prolongs the latency period and impedes mammary tumor growth. To determine the role of GRP78 on endogenous tumor growth, the Grp78+/− mice were crossed with the transgenic mice (MMTVPyVT) expressing the polyoma middle T oncogene (PyT) driven by the murine mammary tumor viral promoter (Guy et al, Mol. Cell. Biol., 12:954-61, 1992). Cohorts of Grp78+/+, PyT mice, Grp78+/−, PyT mice, Grp78+/+ mice, and Grp78+/− mice were monitored for the time of appearance and size of the primary mammary tumors. In the Grp78+/+, PyT mice, most tumors were first detectable between week 8 and 10, whereas most tumors in the Grp78+/−, PyT mice became detectable between week 10 and 12 (FIG. 6A). Neither the Grp78+/+ nor Grp78+/− mice without the PyT allele developed any tumor (FIG. 6A). The growth of the primary tumors in mice bearing the PyT oncogene was monitored for 15 weeks. Statistical analysis indicated that the tumor growth was significantly reduced in the Grp78+/−, PyT mice compared to the Grp78+/+, PyT mice, p<0.001 (FIG. 6B), with the mean tumor volume at week 15 reduced from 201 mm³ for Grp78+/+ mice to 92 mm³ for Grp78+/− mice, a reduction of 109 mm³ [95% Confidence Intervals (CI) 60-159 mm³]. In this mammary tumor model, which is noted for its high penetrance, the mice also developed smaller tumors in subsequent weeks. The size and number of these tumors were also reduced in the Grp78 heterozygous mice. In contrast, the size and morphology of major organs in all four groups of mice (Grp78+/+, Pyr; Grp78+/−, PyT; Grp78+/+; Grp78+/−) were comparable (FIG. 2C).

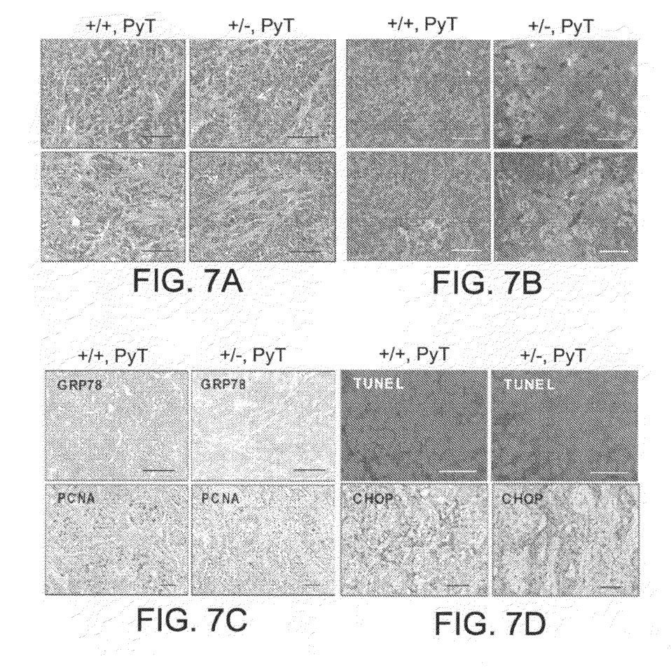

Grp78 heterozygosity inhibits tumor proliferation and promotes apoptosis. To understand the mechanisms that contribute to the slower growth rate of tumors of the Grp78+/−, PyT mice, tumor sections were analyzed. Hematoxylin and eosin (H&E) staining of paraffin-embedded tumor sections revealed that while Grp78+/+, PyT tumors exhibited well-vascularized sheets of polygonal and cohesive tumor cells, the Grp78+/−, PyT tumors showed more discohesive tumor cells admixed with dense stromal component (FIG. 7A). The epithelial origin of the tumor cells was confirmed by the demonstration of positive immunofluorescence staining with anti-keratin antibodies in these cells, while immunofluorescence positivity for vimentin, a mesenchymal cell marker, was confined to fibroblasts, which were more prevalent in the Grp78+/−, PyT tumors (FIG. 7B).

Sections of tumors from the Grp78 WT and heterozygous mice were further subjected to PCNA staining to determine whether Grp78 heterozygosity affects tumor proliferation. Strikingly, cell proliferation was reduced in the Grp78+/−, PyT tumors which expressed lower levels of GRP78, as revealed by immunohistochemical staining (IHC) (FIG. 7C). To test whether the lower levels of GRP78 expression in the Grp78+/−, PyT tumors result in enhanced apoptosis, TUNEL assay was performed. In Grp78 WT tumors, there were very few apoptotic tumor cells; in contrast, tumor cells from the Grp78 heterozygous mice showed enhanced apoptosis in general (FIG. 7D, upper panel). Further, in some regions of the Grp78+/−, PyT tumors, IHC staining of CHOP was observed (FIG. 7D, lower panel).

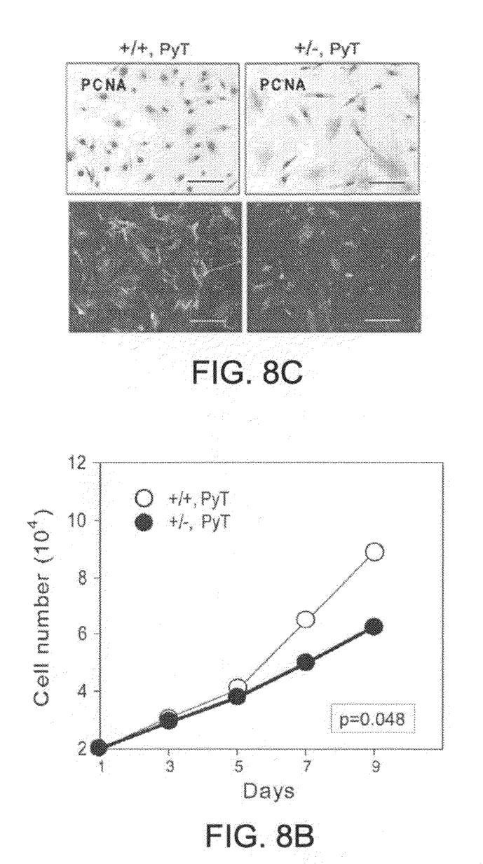

Tumor cells derived from the Grp78+/−, PyT mice grow slower in vitro. Primary tumor cells from the Grp78 WT and heterozygous mice were propagated in vitro. To examine whether decreased proliferation of the Grp78+/−, PyT tumor cells is an intrinsic property of the epithelial tumors cells with reduced GRP78 expression, or due to alterations in the tumor microenvironment in the Grp78 heterozygous mice, direct comparison of the proliferative rates of the cells in culture were performed to eliminate the extrinsic factors such as tumor hypoxia and angiogenesis. The decrease in proliferation of tumor cells from the Grp78+/−, PyT group was evident from substantially lower PCNA labeling as compared to the Grp78+/+, PyT group (FIG. 8A, upper panel). The identity of the epithelial tumor cells in each group was further confirmed by co-staining with fluorescent anti-keratin antibody (FIG. 8A, lower panel). To confirm the staining results, the growth rates of multiple primary cell lines derived from Grp78+/+, PyT and Grp78+/−, PyT tumors were directly monitored (FIG. 8B). The growth rates began to diverge after 5 days of culture. By 9 days of culture, the mean number of tumor cells was significantly decreased from 8.9×10⁴ for the Grp78+/+, PyT group to 6.3×10⁴ for the Grp78+/−, PyT group, a 29% reduction (p=0.048).

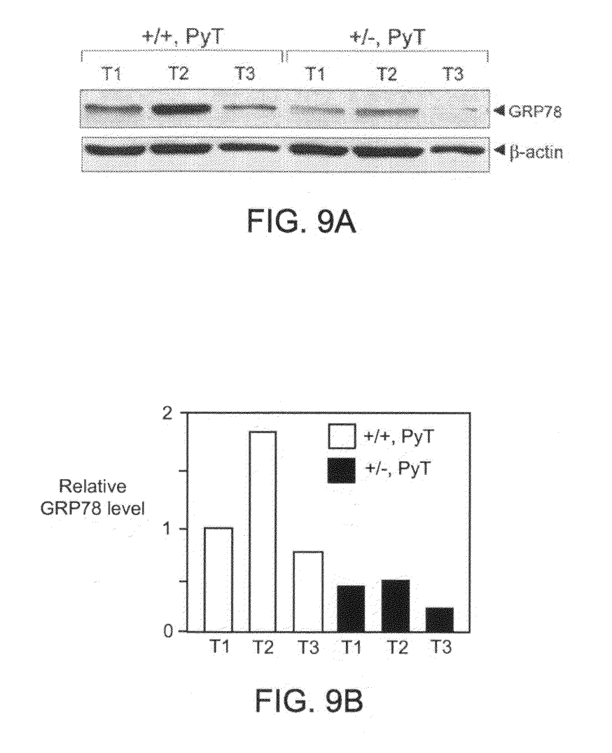

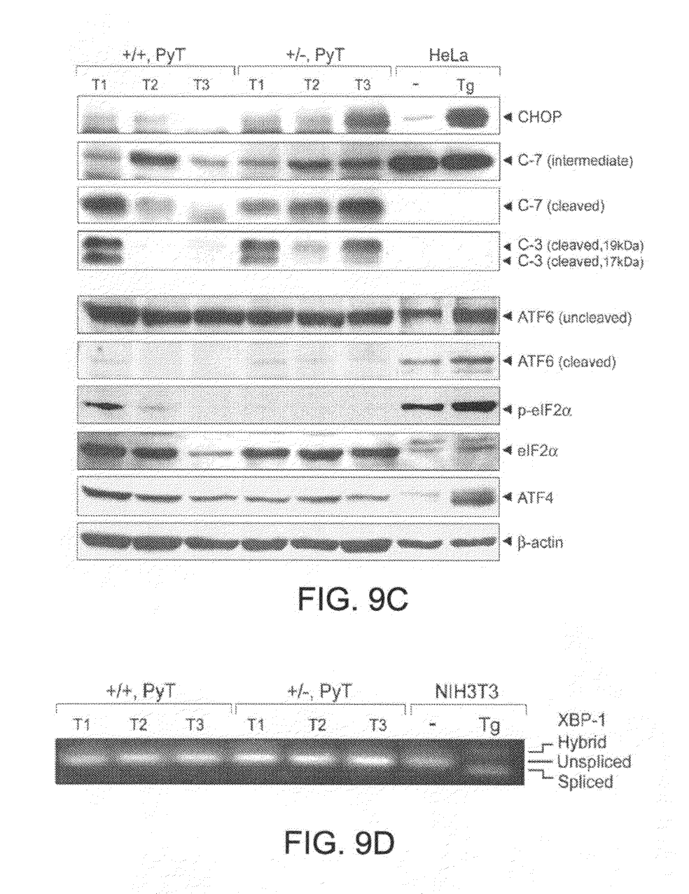

Grp78 heterozygosity upregulates CHOP and caspases in tumors. As an ER stress inducible transcription factor, CHOP has been reported to induce apoptotic cell death by promoting protein synthesis and oxidation in the stressed ER (Marciniak et al, Genes Dev., 18:3066-77, 2004). To extend these observations, Western blot analysis was performed on cell lysates of tumors from the Grp78 WT and heterozygous mice. Tumors of different sizes were examined and, in general, tumors from the Grp78 heterozygous mice showed lower level of GRP78, confirming results obtained from immunohistochemical staining of tumor sections (FIGS. 9A and 9B). CHOP induction was observed in Grp78+/−, PyT tumors, which expressed the lowest level of GRP78 (FIG. 5C). GRP78 is known to bind procaspase-7 (C-7) and block its activation (Reddy et al, J. Biol. Chem., 278:20915-24, 2003; Davidson et al, Cancer Res., 65:4663-72, 2005; and Ermakova et al, Cancer Res., 66:9260-9, 2006). Grp78+/−, PyT tumors of all sizes showed C-7 activation, as evidenced by C-7 cleavage; with strongest activation in tumors expressing the lowest GRP78 level (FIG. 9C). Activation of C-7 was also observed in large Grp78+/+, PyT tumors. Caspase-3 activation, as evidenced by cleavage products, was generally observed in Grp78+/−, PyT tumors, and in large Grp78+/+, PyT tumors (FIG. 9C). The large tumors, in general, displayed necrotic regions which were not observed in medium or small sized tumors.

To examine whether the reduction of GRP78 levels in tumors from the Grp78 heterozygous mice alters the UPR signaling pathways, ATF6 activation was examined by Western analysis for detection of its cleavage products. Compared to the positive control, where the cells were treated with the classical ER stress inducer, thapsigargin, ATF6 activation in both sets of tumors was negligible, albeit a slightly higher level in the Grp78+/−, PyT tumors in general (FIG. 9C). Phosphorylated eIF2a, a downstream target of PERK, was detected in Grp78+/+, PyT but not Grp78+/−, PyT tumors. The level of ATF4, another PERK target, was either not affected or slightly lower in the Grp78+/−, PyT tumors (FIG. 9C). Activation of the IRE1 pathway leads to XBP-1 splicing. In tumors from the Grp78 WT or heterozygous mice, the level of XBP-1 transcript was the same and no splicing was detected (FIG. 9D).

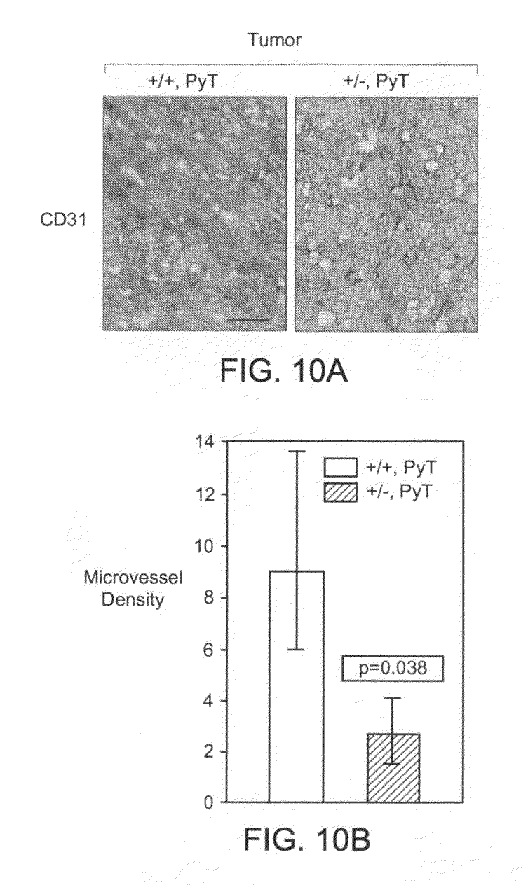

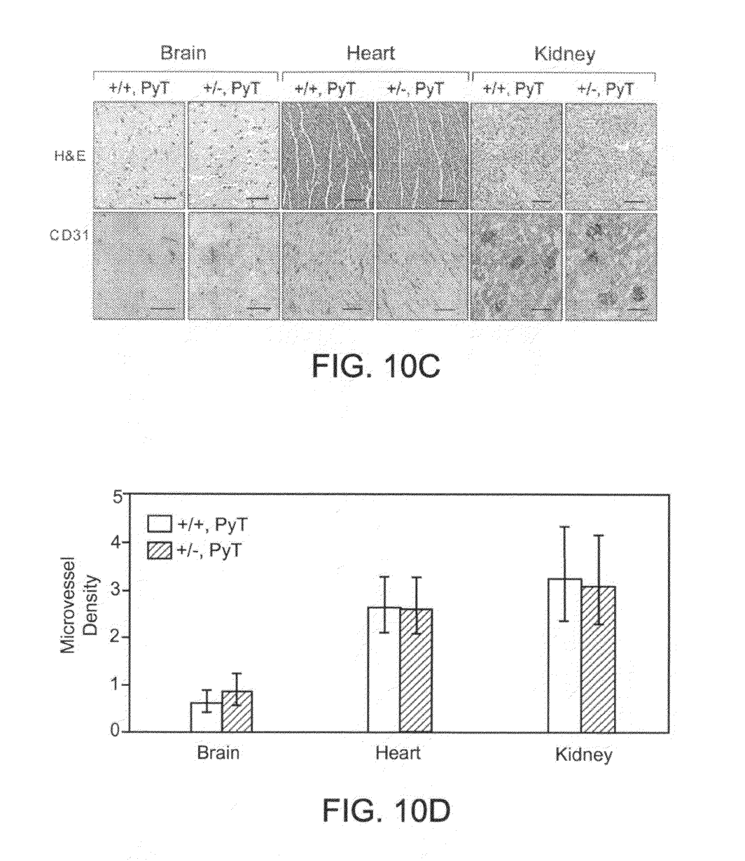

Grp78 heterozygosity inhibits tumor angiogenesis. Another mechanism that may contribute to the slower growth of the Grp78+/−, PyT tumors is that GRP78 is required for tumor angiogenesis. To test this, tumor sections from the Grp78 WT and heterozygous mice were analyzed for MVD through staining with the endothelial cell marker, anti-CD31. In contrast to the well vascularized Grp78+/+, PyT tumors, Grp78+/−, PyT tumors showed a dramatic reduction in MVD (FIG. 10A). Quantitation of the stained tumor vasculature revealed a decrease in the MVD from 9.1 microns² per field for the Grp78+/+, PyT tumors to 2.7 for the Grp78+/−, PyT tumors, a 70% reduction (95% CI 46°/0-83%), p=0.038 (FIG. 10B). In contrast, the vasculature in organs and tissues from the same mice, including brain, heart and kidney, was not affected (FIG. 10C).

1. A method of sensitizing tumor blood vessel cells to a chemotherapeutic agent in a subject, comprising:
a) selecting a subject with a tumor, wherein the cells of the tumor blood vessels are resistant to a first chemotherapeutic agent; and
b) administering to the subject an agent that inhibits expression or activity of GRP78.

2. The method of claim 1, further comprising administering one or more second chemotherapeutic agents to the subject, wherein the second chemotherapeutic agent is the same as or different from the first chemotherapeutic agent to which the tumor blood vessel cells are resistant.

3. The method of claim 1, wherein expression of GRP78 protein is inhibited.

4. The method of claim 1, wherein the activity of GRP78 is inhibited.

5. The method of claim 3, wherein the expression of GRP78 protein is inhibited by inactivating a GRP78 gene or a GRP78 promoter.

6. The method of claim 1, wherein the agent that inhibits expression of GRP78 is selected from the group consisting of an antisense molecule, a triple helix molecule, a ribozyme and an siRNA.

7. The method of claim 6, wherein the siRNA comprises SEQ ID NO:1 or SEQ ID NO:2.

8. The method of claim 1, wherein the agent that inhibits activity of GRP78 is a GRP78 antagonist.

9. The method of claim 8, wherein the GRP78 antagonist is selected from the group consisting of an antibody to GRP78, (−)-epigallocatechin gallate, genistein, salicyclic acid from plants, bacterial AB₅ subtilase cytoxin, and versipelostatin.

10. The method of claim 8, wherein the GRP78 antagonist is a combination of a taxane and doxirubicin.

11. The method of claim 10, wherein the taxane is paclitaxel or docetaxel.

12. The method of claim 1, further comprising administering radiation therapy to the subject.

13. The method of claim 1, wherein the first chemotherapeutic agent is selected from the group consisting of etoposide, CPT-11 and temozolomide.

14. The method of claim 2, wherein the second chemotherapeutic agent is selected from the group consisting of CPT-11, temozolomide (TMZ), bleomycin, carboplatin, chlorambucil, cisplatin, colchicine, cyclophosphamide, daunorubicin, dactinomycin, diethylstilbestrol doxorubicin, etoposide, 5-fluorouracil, floxuridine, melphalan, methotrexate, mitomycin, 6-mercaptopurine, teniposide, 6-thioguanine, vincristine and vinblastine.

15. The method of claim 1, wherein the tumor is a glioblastoma.

16. The method of claim 8, wherein the GRP78 antagonist is not kringle 5 or a derivative of kringle 5.

17. A method of reducing tumor microvessel density in a subject, comprising:
a) selecting a subject with a tumor, wherein the subject is in need of reduction in tumor microvessel density; and
b) administering to the subject an agent that inhibits expression or activity of GRP78.

18. The method of claim 17, wherein expression of GRP78 protein is inhibited.

19. The method of claim 17, wherein the activity of GRP78 is inhibited.

20. The method of claim 18, wherein the expression of GRP78 protein is inhibited by inactivating a GRP78 gene or a GRP78 promoter.

21. The method of claim 17, wherein the agent that inhibits expression of GRP78 is selected from the group consisting of an antisense molecule, a triple helix molecule, a ribozyme and an siRNA.

22. The method of claim 21, wherein the siRNA comprises SEQ ID NO:1 or SEQ ID NO:2.

23. The method of claim 17, wherein the agent that inhibits activity of GRP78 is a GRP78 antagonist.

24. The method of claim 23, wherein the GRP78 antagonist is selected from the group consisting of an antibody to GRP78, (−)-epigallocatechin gallate, genistein, salicyclic acid from plants, bacterial AB₅ subtilase cytoxin, and versipelostatin.

25. The method of claim 23, wherein the GRP78 antagonist is a combination of a taxane and doxirubicin.

26. The method of claim 25, wherein the taxane is paclitaxel or docetaxel.

27. The method of claim 17, further comprising administering radiation therapy to the subject.

28. The method of claim 17, further comprising administering a chemotherapeutic agent to the subject.

29. The method of claim 28, wherein the chemotherapeutic agent is selected from the group consisting of CPT-11, temozolomide (TMZ), bleomycin, carboplatin, chlorambucil, cisplatin, colchicine, cyclophosphamide, daunorubicin, dactinomycin, diethylstilbestrol doxorubicin, etoposide, 5-fluorouracil, floxuridine, melphalan, methotrexate, mitomycin, 6-mercaptopurine, teniposide, 6-thioguanine, vincristine and vinblastine.


SEQUENCE LISTING (8 sequences)

SEQ ID NO: 1 - DNA, 21 bp, Artificial Sequence
  siRNA GRP78A
ggagcgcauugauacuagatt

SEQ ID NO: 2 - RNA, 21 nt, Artificial Sequence
  siRNA GRP78B
aagaaaagcugggagguaaac

SEQ ID NO: 3 - RNA, 21 nt, Artificial Sequence
  siRNA Control A
ggagaagaauagcaacgguaa

SEQ ID NO: 4 - DNA, 21 bp, Artificial Sequence
  siRNA Control B
aaggugguuguuuuguucatt

SEQ ID NO: 5 - DNA, 20 bp, Artificial Sequence
  Synthetic primer
tagaaagaaagcccggatga

SEQ ID NO: 6 - DNA, 20 bp, Artificial Sequence
  Synthetic primer
aaagggaggctggtaaggaa

SEQ ID NO: 7 - DNA, 21 bp, Artificial Sequence
  Synthetic primer
gaaccaggagttaagaacacg

SEQ ID NO: 8 - DNA, 20 bp, Artificial Sequence
  Synthetic primer
aggcaacagtgtcagagtcc